(19) United States
(12) Patent Application Publication
(10) Pub. No.: US 2022/0160263 A1
(43) Pub. Date: May 26, 2022

(54) OPTICAL FILTER DEVICE, SYSTEM, AND METHOD FOR IMPROVED OPTICAL REJECTION OF OUT-OF-BAND WAVELENGTHS

(71) Applicant: Profusa, Inc., Emeryville, CA (US)

(72) Inventor: Clayton LEPAK, Berkeley, CA (US)

(73) Assignee: Profusa, Inc., Emeryville, CA (US)

(21) Appl. No.: 17/669,828

(22) Filed: Feb. 11, 2022

Related U.S. Application Data
(63) Continuation of application No. PCT/US2020/047188, filed on Aug. 20, 2020.
(60) Provisional application No. 62/889,539, filed on Aug. 20, 2019.

(51) Int. Cl. A61B 5/1459; G02B 5/20

(52) U.S. Cl. CPC A61B 5/1459; A61B 5/14532; G02B 5/20

(57) ABSTRACT

An optical filter device, system, and method for improved optical rejection of out-of-band wavelengths is disclosed. For example, an analyte detection system is provided that includes an excitation light source for illuminating an implantable sensor and an optical detector for collecting emission light from the implantable sensor. Further, the analyte detection system includes an optical filter device arranged between the implantable sensor and the optical detector, wherein the optical filter device provides high optical rejection of out-of-band wavelengths of the emission light.

### CROSS REFERENCE TO RELATED APPLICATIONS

This application is a continuation of International Patent Application No. PCT/US2020/047188, filed Aug. 20, 2020, which claims priority to U.S. Provisional Patent Application No. 62/889,539, filed Aug. 20, 2019, the entire disclosure of each of which is hereby incorporated by reference.

### TECHNICAL FIELD

The presently disclosed subject matter relates generally to optical band-pass filters and more particularly to an optical filter device, system, and method for improved optical rejection of out-of-band wavelengths.

### BACKGROUND

In the management of many conditions, the regular measurement of analytes in vivo is desirable. It has been a long-standing objective of both medical science and the military to implant sensors inside the human body that continuously and accurately determine changes in physiologic, metabolic, or fatigue status; measure the concentration of biothreat or therapeutic agents in vivo; and provide early detection of disease prior to the onset of symptoms. Such sensors are preferably implanted though a non- or minimally-invasive procedure, require minimal user maintenance, and are able to operate for months to years.

For example, measurement of glucose in the blood can improve the ability to correctly dose insulin in diabetic patients. Furthermore, it has been demonstrated that in the long term care of the diabetic patient, better control of blood glucose levels can delay, if not prevent, the onset of retinopathy, circulatory problems and other degenerative diseases often associated with diabetes. Thus there is a need for reliable and accurate self-monitoring of blood glucose levels by diabetic patients.

Currently, biosensors exist that can be implanted in tissue. For example, biosensors exist that can be implanted a few millimeters under the skin. In some such sensors, luminescent dyes are used to measure the concentration of an analyte of interest (e.g., oxygen, glucose, lactate, carbon dioxide (CO₂), pH). For example, the intensity of certain luminescent dye can modulate based on the amount of analyte present, such that the intensity of the emission light can be correlated to the analyte concentration. However, intensity-based systems can be challenging because the detector (or reader) is subject to potential sources of error and noise that make it difficult to get an accurate analyte measurement. Implantable sensors and associated components are described in U.S. Pat. Nos. 9,375,494; 10,117,613; 10,219,729; and 10,717,751 and U.S. Patent Application Pub. Nos. 2016/037455, the entire disclosure of each of which is hereby incorporated by reference in its entirety.

Because the optical power of a fluorophore excitation source is often orders of magnitude stronger than the resulting fluorescence emission, using an optical filter to separate the excitation light from the emission light has certain challenges. Namely, the cutoff wavelengths (or filter window) for optical band-pass filters are dependent on the angle of incidence of the incident light. As angle of incidence increases, the filter window shifts to shorter wavelengths (i.e., blue shifts). In the case of fluorophore excitation and emission, this blue shift causes the optical filter window for the emission to shift towards the excitation light source. Accordingly, when relying on intensity-based measurements, a challenge exists for providing an optical filter that can reject excitation light at orders of magnitude greater than emission light power at the worst-case angle of incidence of the system.

### BRIEF DESCRIPTION OF THE DRAWINGS

Having thus described the presently disclosed subject matter in general terms, reference will now be made to the accompanying drawings, which are not necessarily drawn to scale, and wherein:

FIG. 1 illustrates a block diagram of an example of the presently disclosed analyte detection system including an optical filter device configured to provide high optical rejection of out-of-band wavelengths, according to an embodiment.

FIG. 2 illustrates a block diagram of an optical filter device of an analyte detection system, according to an embodiment.

FIG. 3 illustrates a block diagram of an optical filter device, according to an embodiment.

FIG. 4 illustrate a block diagram of an optical filter device, according to an embodiment.

FIG. 5 illustrates a block diagram of an optical filter device, according to an embodiment.

FIG. 6 is a bar graph showing experimental results comparing the emission-to-excitation ratio of various optical filter configurations.

### DETAILED DESCRIPTION

Embodiments described herein generally relate to an optical filter device and/or system and method for improved optical rejection of out-of-band wavelengths. According to some embodiments, an analyte detection system includes an excitation light source for illuminating an implantable sensor and an optical detector for collecting emission light from the implantable sensor. An optical filter device can be operable to reject out-of-band wavelengths, for example originating from the excitation light source, while allowing a signal of interest, for example originating from the implantable sensor, to be received by the optical detector. According to some embodiments, optical filter devices described herein can be operable to provide high optical rejection of out-of-band wavelengths of light from an optical band-pass filter even when the incident light on the filter is scattered light that strikes the surface of the filter at angles of incidence ranging from nearly +90 degrees to −90 degrees. Thus, optical filter devices described herein are capable of providing efficient optical filtering of uncollimated fluorophore excitation light from uncollimated fluorophore emission light in a simple, stray light-insensitive, compact, manufacturable form-factor that is suitable for use in, for example, a wearable detection device.

An analyte detection system, according to some embodiments, include an optical filter device that includes one or more angular filters in combination with one or more optical filters. Optical filter devices described herein typically include at least three layers (e.g., a stack of bandpass and angular filters). Such optical filter devices are capable of substantially rejecting the excitation light signal while transmitting the emission light signal. In one example, the optical filter device includes, in order, a first angular filter, an optical bandpass filter, and a second angular filter. In another example, the optical filter device includes, in order, a first optical bandpass filter, an angular filter, and a second optical bandpass filter. In yet another example, the optical filter device includes, in order, a first angular filter, a first optical bandpass filter, a second angular filter, and a second optical bandpass filter.

Embodiments described herein can include an optical filter device that can reject excitation light at orders of magnitude greater than emission light power at the worst-case angle of incidence of the system.

In some embodiments, an analyte detection system that includes an optical filter device is implemented in a wearable detection device.

In some embodiments, an analyte detection system including an optical filter device can be physically scalable to incorporate into a wearable detection device. Namely, the optical filter device may be provided in a form factor that is suitable for a wearable detection device.

Some embodiments described herein relate to a method that includes subjecting a diffuse optical signal to an optical filter device to reject components associated with an excitation light source, while passing components associated with an emission signal. An excitation light source can be operable to illuminate a sensor disposed in a highly scattering environment, such as tissue. The scattering environment can cause light from the excitation source to scatter and reflect back towards the excitation light source at a wide range of angles. The sensor can be operable to absorb a portion of the excitation light and emit the emission signal at a different, typically higher, wavelength. Light that exits the scattering environment (the diffuse optical signal) can therefore include components associated with the excitation light source and components associated with emissions from the sensor.

The method can include subjecting the diffuse optical signal to a first angular filter to produce a first filtered optical signal. The first angular filter can be configured to reject components of the diffuse optical signal that have an angle of incidence outside a predefined range (e.g., greater than 20 degrees and/or less than −20 degrees). Components that pass the first angular filter (e.g., a first filtered optical signal) can be subjected to a bandpass filter that is configured to reject components of the first filtered optical signal that that have an angle of incidence less than 30 degrees (and/or greater than −30 degrees) and a wavelength shorter than a first predefined thereshold. Components that pass the bandpass filter (e.g., a second filtered optical signal) can be subjected to a second angular filter that is configured to reject components of the second filtered optical signal that have an angle of incidence greater than 20 degrees (and/or less than −20 degrees). Components that pass the second angular filter (e.g., a third filtered optical signal) can be sensed by a detector. The components sensed by the detector can have a very high signal to noise (or emission to excitation) ratio.

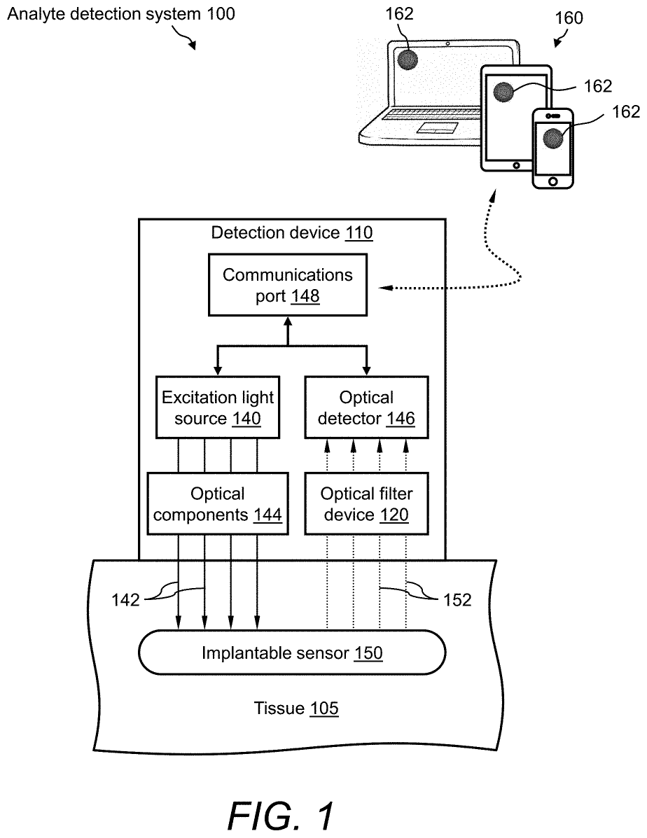

FIG. 1 is a block diagram an analyte detection system 100, according to an embodiment, that including an optical filter device providing high optical rejection of out-of-band wavelengths. The analyte detection system 100 and optical filter device can be used for reading an implantable sensor and determining an analyte value.

The analyte detection system 100 includes a detection device 110 that can be positioned adjacent to an implantable sensor 150 implanted in tissue 105. For example, implantable sensor 150 may be implanted a few millimeters (e.g., 1-10 mm) under the skin of the user and the detection device 110 can be positioned outside the tissue and over the implantable sensor.

Implantable sensor 150 may be, for example, an analyte-sensing fluorescent sensor. When implanted in tissue 105, implantable sensor 150 is in good contact (close proximity) to blood vessels and has direct access to of interstitial fluid and can therefore be operable to measure various biological analytes. Implantable sensor 150 includes analyte-sensing dye. The analyte-sensing dye in implantable sensor 150 can be an analyte-specific dye for targeting the analyte of interest. Examples of analytes of interest may include, but are not limited to, oxygen, reactive oxygen species, glucose, lactate, pyruvate, cortisol, creatinine, urea, sodium, magnesium, calcium, potassium, vasopressin, hormones (e.g., Luteinizing hormone), pH, CO₂, cytokines, chemokines, eicosanoids, insulin, leptins, small molecule drugs, ethanol, myoglobin, nucleic acids (RNAs, DNAs), fragments, polypeptides, single amino acids, and the like. In one example, implantable sensor 150 may be a glucose sensor and therefore the analyte-sensing dye is a glucose-sensing dye.

Detection device 110 is an optical device that includes an excitation light source 140 operable to illuminate and excite the implantable sensor 150, an optical detector 146 operable to receive signals emitted by the implantable sensor 150, and an optical filter device 120 that provides high optical rejection (e.g., 10⁻⁵, 10⁻⁶, or 10⁻⁷ optical rejection) of out-of-band wavelengths (e.g., noise associated with the excitation light source 140). Detection device 110 further includes certain optical components 144 and a communications port 148. In some embodiments, detection device 110 may include a power source (not shown), such as a battery. Detection device 110 is designed to be fitted against the surface of the skin. Detection device 110 may be implemented using a printed circuit board (PCB), a flexible PCB, or other flexible substrate. Detection device 110 may be, for example, a wearable detection device provided as a patch that can be placed on the surface of the skin (i.e., tissue 105) in close proximity to implantable sensor 150.

Excitation light source 140 is arranged to transmit excitation light 142 from the surface of the skin, through the tissue 105, and to implantable sensor 150. The excitation light 142 from excitation light source 140 is within the excitation wavelength range of any analyte-sensing dye of implantable sensor 150. Suitable excitation light sources may include, but are not limited to, lasers, semi-conductor lasers, light emitting diodes (LEDs), and organic LEDs. Optical components 144 may include any types of components (e.g., optical filters) needed in detection device 110 for conditioning excitation light source 140.

The optical detector 146 is operable to detect emission light 152 from the analyte-sensing dye of implantable sensor 150 that has passed through and exited the tissue 105. Namely, optical detector 146 detects emission light 152 in the emission wavelength of the analyte-sensing dye of implantable sensor 150. Suitable optical detectors may include, but are not limited to, photodiodes, complementary metal-oxide-semiconductor (CMOS) detectors, and charge-coupled device (CCD) detectors.

As discussed in further detail herein, optical detector 146 can be filtered using optical filter device 120 such that the optical detector 146 is operable to measure the optical signals emitted within the desired wavelength ranges (e.g., the emission wavelength range) and such that optical filter device 120 provides high optical rejection of out-of-band wavelengths (e.g., the excitation wavelength band) the as compared with conventional optical detection devices.

In use, the implantable sensor 150 is excited at its excitation wavelength via excitation light 142. Then, implantable sensor 150 absorbs the excitation light 142 and emits longer wavelength emission light 152. Then, optical filter device 120 rejects the excitation light 142 allowing for the emission light 152 to be measured accurately by optical detector 146. As discussed in further detail herein, however, because tissue is a highly scattering environment, portions of the excitation light 142 strike the optical filter device at a wide range of angles of incidence (e.g., from −89 degrees to 89 degrees). Known bandpass filters may be ineffective to discriminate between emission light 152 and high angle of incidence excitation light. Optical filter device 120, therefore, may include, for example, an arrangement or stack of one or more optical components.

Detection device 110 can include built-in electronic processing device(s) (not shown) and/or data storage (not shown). In such embodiments, the processing capability of analyte detection system 100 can be completely or partially on board detection device 110 that is located on the surface of the skin. In addition or alternatively, the processing capability of analyte detection system 100 is external to detection device 110 that is located on the surface of the skin. Accordingly, communications port 148 is provided between detection device 110 and a separate computing device 160, wherein computing device 160 may be used for processing any information from detection device 110. Computing device 160 may be any type of computing device, such as a desktop computer, a laptop computer, a tablet device, a mobile phone, a smartphone, a centralized server or cloud computer, and the like. In this example, communications port 148 facilitates a wired and/or wireless communications link from excitation light source 140 and/or optical detector 146 to, for example, computing device 160. For example, communications port 148 may be a wired communications port, such as a USB port, and/or a wireless communications port that uses, for example, WiFi and/or Bluetooth® technology.

Computing device 160 may use a desktop application 162 or mobile app 162 to process any information from implantable sensor 150. Namely, desktop application 162 or mobile app 162 may include any software and/or hardware components for processing any information from implantable sensor 150. While detection device 110 may include battery power, in other embodiments, computing device 160 supplies power to detection device 110.

In one example, computing device 160 may be used to activate excitation light source 140, wherein excitation light source 140 emits excitation light 142 and illuminates the analyte-sensing dye in implantable sensor 150, wherein the analyte-sensing dye has a certain absorption spectrum and a certain emission spectrum. Then, optical detector 146 collects emission light 152 from implantable sensor 150 that passes through optical filter device 120 and wherein optical filter device 120 provides high optical rejection of out-of-band wavelengths of emission light 152. Then, computing device 160 collects information from optical detector 146, wherein optical detector 146 converts optical signals received from implantable sensor 150 to an electrical signal output. The measured intensity of emission light 152 correlates to an analyte value. For example, in an implantable glucose sensor 150 the measured intensity of emission light 152 (i.e., fluorescence) correlates to the amount or concentration of glucose present. Generally, excitation light 142 is orders of magnitude stronger than emission light 152. Accordingly, optical filter device 120 is used to separate excitation light 142 and emission light 152. Namely, optical filter device 120 is used to reject excitation light 142 as much as possible to increase the signal-to-noise ratio of light that illuminates the optical detector 146. As described in further detail herein, the optical filter device 120 is particularly well suited to efficiently reject excitation light for fluorophores with short stoke-shifts, even in highly scattering environments such that the acceptable range of angle of incidence on the optical filter device 120 is higher than is possible with known filtering techniques used for short stokes-shift fluorophores. For example, particularly for fluorophores where the excitation peak is very close to the emission peak (e.g., 50, 30, 25, or 15 nm or less) discriminating between an emission signal and backscattered excitation signal at an off-angle can be difficult using known filters and filtering methods. Embodiments described herein are capable of high optical rejection (e.g., greater than 10⁻⁵) of out-of-band light at high angles of incidence (e.g., outside of +/−30 degrees), which may not achievable according to known methods suitable for detecting short stoke-shift emissions from an implantable sensor.

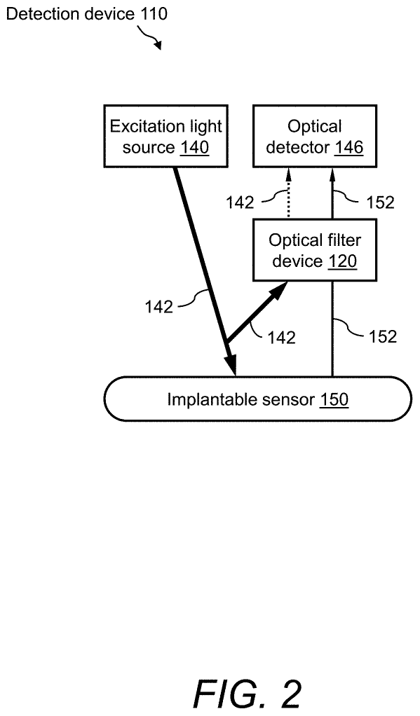

FIG. 2 is a block diagram of the detector device 110 shown and described above with reference to FIG. 1, showing additional details of the optical filter device 120. The optical power of the excitation light source 140, configured to excite a fluorophore is often orders (e.g., 1, 2, or 3) of magnitude stronger than the resulting fluorescence emission. Therefore, it is desirable for the optical filter device 120 to be designed to reject excitation light at orders of magnitude greater than emission light power at the worst-case angle of incidence of the system. Namely, the cutoff wavelengths (or filter window) for optical band-pass filters are often dependent on the angle of the incident light. As angle of incidence increases, the filter window shifts to shorter wavelengths (i.e., blue shifts). In the case of fluorophore excitation and emission, this blue shift causes the optical filter window for the emission to shift towards the excitation light source, in some cases rendering the optical band-pass filters ineffective (allowing greater than 50%, 75%, 90% etc. of out-of band light to pass) at rejecting light in the excitation bandwidth range that has a high angle of incidence (e.g., outside of +/−20, 25, 30, 35, 45 degrees). Accordingly, optical filter device 120 of analyte detection system 100, which may rely on intensity-based measurements, can be operable to reject excitation light at orders of magnitude greater than emission light power at the worst-case angle of incidence of the system.

FIG. 2 shows excitation light 142 striking implantable sensor 150. Additionally, excitation light 142 reaches the optical filter device 120 (e.g., due to diffuse reflectance and/or back scatter) across a wide range of angles of incidence (e.g., from −89 degrees to 89 degrees). In response to excitation light 142, implantable sensor 150 produces emission light 152. Typically the optical detector 146 is positioned over the implantable sensor 150, such that much of the emission light 152 is substantially normal to optical filter device 120.

As discussed in further detail herein, the optical filter device 120 is designed to reject excitation light that is orders of magnitude more intense than emission light power at the worst-case angle of incidence of the system (e.g., +/−89 degrees). Thus optical filter device 120 substantially rejects excitation light 142 that reaches the optical filter device at or near 0 degrees angle of incidence (e.g., normal excitation light) and at high angles of incidence, while at the same time transmitting emission light 152. For example, the emission-to-excitation ratio of emission light 152-to-excitation light 142 at the output of optical filter device 120 is large, according to some embodiments>200.

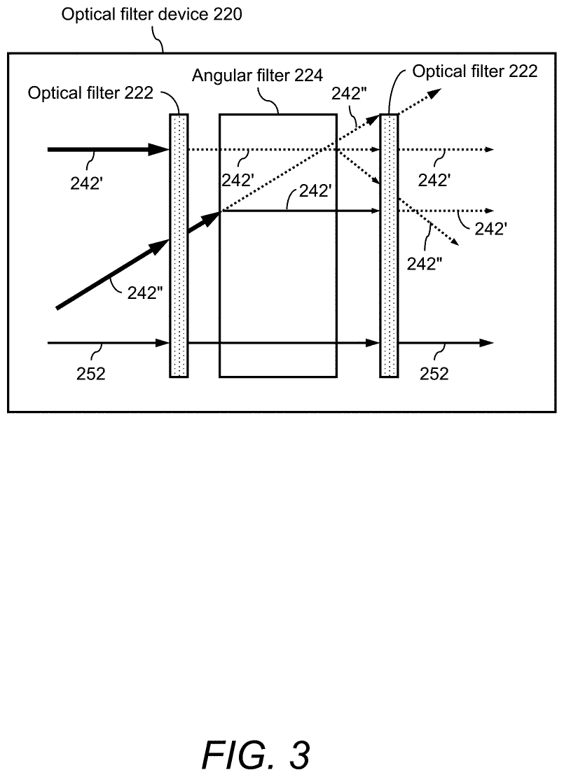

Generally, the presently disclosed analyte detection system 100 provides an optical filter device 120 that includes one or more angular filters in combination with and alternating with one or more optical filters in order to substantially reject the excitation light signal and while transmitting the emission light signal. The optical filter device 120 typically includes at least three layers, as experimental results have demonstrated that two or few layers provides dramatically inferior rejection of out-of-band light. In some instances, as compared to a two-layer optical filter device, a three-layer optical filter device can increase the signal-to-noise ratio by a factor of >350. For example, as shown according to the embodiment depicted in FIG. 3, an optical filter 220 includes, in order, a first optical filter 222, an angular filter 224, and a second optical filter 222.

Optical filter 220 allows filtering of out-of-band light from a diffuse source (e.g., tissue). First and second optical filters 222 can be thin film optical bandpass filters. First and second optical filters 222 may be, for example, the 707 nm filter (p/n PROF-0016) available from Semrock, a unit of IDEX Health & Science, LLC (Rochester, N.Y.). An angular filter allows normal light (light striking the angular filter 224 at or near a 0 degree angle of incidence, light striking the angular filter 224 at an angle of incidence between +10 degrees and −10 degrees, light striking the angular filter 224 at an angle of incidence between +20 degrees and −20 degrees, etc.) to pass through while preventing light at high angle (e.g., light having an angle of incidence outside of 30 degrees) from passing through. Accordingly, angular filter 224 provides a certain angular rejection of light. Angular filter 224 may be, for example, a fiber optic plate (FOP). An FOP is an optical device formed of a bundle of micron-diameter fibers. An FOP directly conveys light or image incident on its input surface to its output surface. Examples of FOPs suitable for optical filter device 220 may include, but are not limited to, the SCHOTT® Fiber Optic Faceplates available from SCHOTT North America, Inc. (Southbridge, Mass.) and the FOPs available from Hamamatsu Corporation (Bridgewater, N.J.). In another example, angular filter 224 may be series of apertures.

While the aforementioned example components may be suitable for glucose-specific dye, more generally the components of optical filter device 220 may be:

### Optical Filters:

Bandpass Wavelengths in the following range: 400 nm-1600 nm

Substrate: Glass, plastic, other transparent materials.

Optical Density (OD) outside of passband, specifically near excitation wavelengths: >4 OD

Optical Transmission in pass-band: >1%

Steep cut on/off edges: <30 nm cutoff width

### Fiber Optic Plates (FOP)

Numerical Aperature: 0.5-0.05

Normal incident transmission: >1%

Stray Light Control: EMA glass or equivalent to prevent crosstalk between fibers

High angle light rejection at: OD>4

Apertures (single or array)

High angle light rejection at: OD>4

Normal incident transmission: >1%

Lenses (single or array)+system of apertures

Numerical Aperture: 0.5-0.05

High angle light rejection at: OD>4

Normal incident transmission: >1%

In operation, the specifications (e.g., wavelength pass band) of first optical filter 222, angular filter 224, and second optical filter 222 are selected such that emission light 252 (at a predefined wavelength) passes through the arrangement in a substantially unfiltered fashion and such that excitation light 242 (at a different, lower, predefined wavelength) is substantially rejected. With respect to rejecting excitation light 242, both a normal component of excitation light 242 (e.g., normal excitation light 242′) and a high angle component of excitation light 242 (e.g., high angle excitation light 242″) reaches optical filter device 220. First optical filter 222 substantially filters out normal excitation light 242′ such that a negligible amount (e.g., >10⁻⁵, 10⁻⁶, 10⁻⁷⁻) of normal excitation light 242′ passes down the line and reaches the output of optical filter device 220. However, high angle excitation light 242″ (e.g., light having an angle of incidence greater than 25, 30, 35, 45, degrees, etc. and/or less than −25, −30, −35, −45 degrees etc.) passes through first optical filter 222 and reaches angular filter 224. Similarly stated, the first optical filter 222 may be ineffective (may be operable to reject less than 50% of) light within the excitation bandwidth range that strikes the first optical filter 222 at high angles of incidence. Angular filter 224 substantially filters out high angle excitation light 242″ such that a negligible amount of high angle excitation light 242″ passes down the line and reaches the output of optical filter device 220. However, when high angle excitation light 242″ reaches the interface of angular filter 224 a new normal excitation light 242′ component may be formed that passes on to second optical filter 222. Second optical filter 222 substantially filters out this normal excitation light 242′ such that only a negligible amount thereof reaches the output of optical filter device 220. In this manner, optical filter device 220 is used to substantially reject any normal and high angle components of excitation light 242 while transmitting emission light 252.

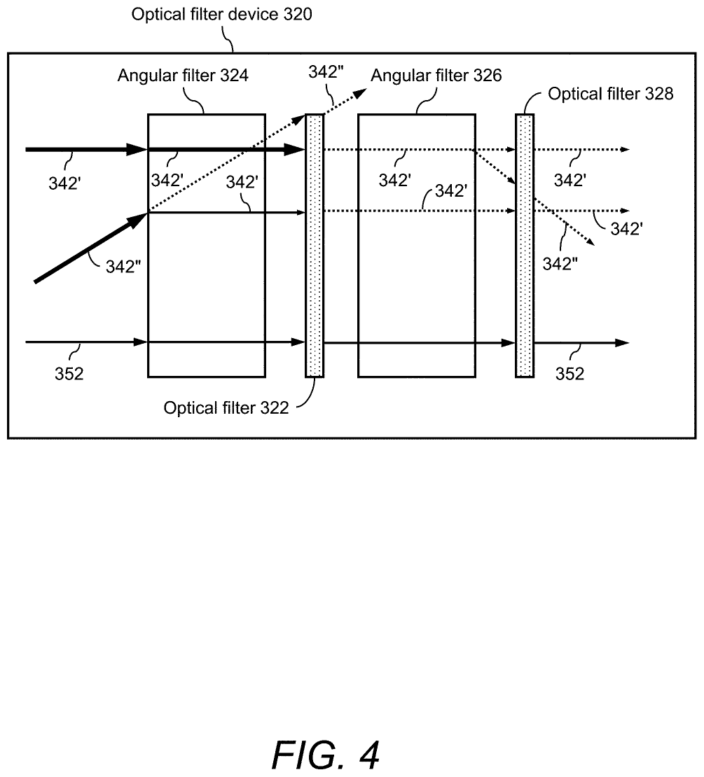

According to another embodiment shown in FIG. 4, an optical filter device 320 includes, in order, a first angular filter 324, a first optical bandpass filter 322, a second angular filter 326, and a second optical bandpass filter 328. Namely, optical filter device 320 shown in FIG. 4 is substantially the same as optical filter device 320 shown in FIG. 3 except for the addition of another angular filter 324 in advance of the first optical bandpass filter 322. First angular filter 324 provides an additional level of filtration. For example, to achieve a certain desired level of optical rejection, additional stages (e.g., first angular filter 324) can be added to increase performance.

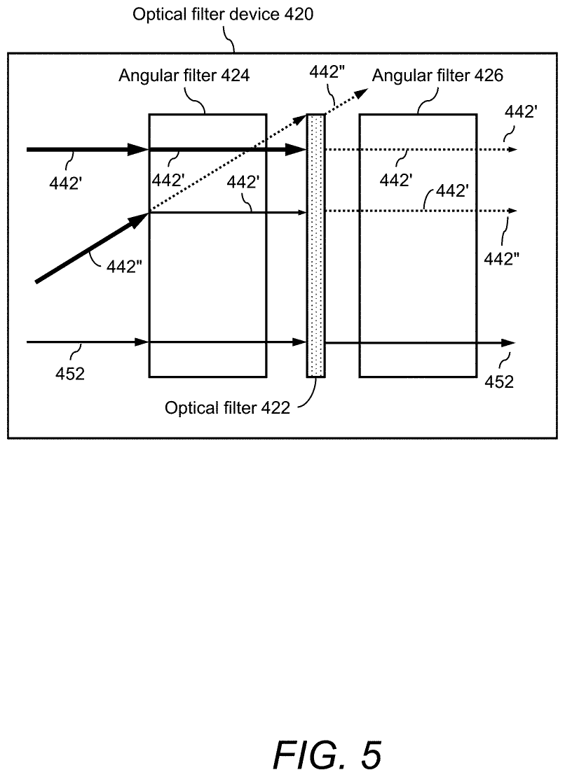

According to another embodiment shown in FIG. 5, an optical filter device 420 includes, in order, a first angular filter 424, an optical bandpass filter 422, and a second angular filter 426. Namely, optical filter device 420 shown in FIG. 5 is substantially the same as optical filter device 320 shown in FIG. 3 except instead of two optical filters and one angular filter, optical filter device 420 includes two angular filters 424, 426 and one optical filter 422.

Optical filter devices are not limited to the number and order of components shown with reference to FIGS. 3-5. These configurations are exemplary only. An optical filter device may include any number of one or more angular filters in combination with two or more optical filters and in any order to substantially reject the excitation light signal while transmitting the emission light signal. Frequently, but not necessarily, angular filters and optical filters alternate in the stack of the optical filter device. However, there may be a balance of the number and arrangement of components without reducing significantly the signal-to-noise (SNR) of optical filter device.

An embodiment includes a wearable detection device which includes a housing bottom and a housing top. A housing bottom may include a housing window, wherein housing bottom is the portion of wearable detection device that is placed against the user's skin. In an aspect, a temperature detector may be included to detect the temperature of the user's skin. The wearable detection device may include a main printed circuit board (PCB) and a skin temp PCB, wherein skin temp PCB may be in thermal contact with the temperature detector and may process skin temperature information from the temperature detector. The main PCB may include a plurality of LEDs and an optical detector. The optical detector is one example of optical detector 146 shown in FIG. 1 and FIG. 2.

In an embodiment, the wearable detection device may also include a processor, which may be the master controller that is used to manage the overall operations of wearable detection device. The processor may be any standard controller or microprocessor device that is capable of executing program instructions. Further, a certain amount of data storage may be associated with the processor. The main PCB may include any other components that may be useful in wearable detection device, such as, but not limited to, a communications interface. In one example, wearable detection device can be used to report out the user's glucose level periodically, such as every few minutes.

In an embodiment, the wearable detection device may include a first dual bandpass filter (e.g., configured to pass optical signals associated with multiple fluorescent dyes having different emission spectra), a first FOP, a second dual bandpass filter, and a second FOP, which may be arranged in a stack. This stack of first dual bandpass filter, first FOP, second dual bandpass filter, and second FOP is one example of the presently disclosed optical filter device 120 that provides high optical rejection of out-of-band wavelengths. More particularly, this stack is one example of optical filter device 320 shown in FIG. 4. Namely, dual bandpass filters is an example of an optical filter(s) 322, 328 of optical filter device 320 of FIG. 4 and FOPs are an example of an angular filter(s) 324, 326 of optical filter device 320 of FIG. 4.

In an embodiment, the wearable detection device may include a battery for supplying power to the active components thereof. The battery may be a rechargeable or non-rechargeable battery.

In one example, the wearable detection device has an overall length of about 3 cm, and overall width of about 2 cm, and an overall thickness or height of about 1 cm. Each of the dual bandpass filters may be, for example, about 1 mm thick. Each of the FOPs may be, for example, from about 0.5 mm to about 1 mm thick. Accordingly, the entire stack may be, for example, from about 2 mm to about 4 mm thick. Additionally, the stack may be, for example, about 4 mm square. In one example, the wearable detection device may be held on the user's skin using an adhesive patch. The housing window of the wearable detection device may be positioned in relation to an implantable sensor, such as implantable sensor 150, in order to capture optical readings therefrom.

In other embodiments, instead of using discrete components, such as the stack of dual bandpass filters and FOPs, optical filter device 120 and optical detector 146 can be provided as an integrated component formed entirely using silicon manufacturing methods. For example, an optical detector is provided at the wafer and die level. Then, at the wafer level, the die are coated with a filter material. Then, a series of lenses or angular filters are deposited on the filter. Then, the wafer is diced to form individual integrated circuit (IC) devices that include both optical filter device 120 and optical detector 146.

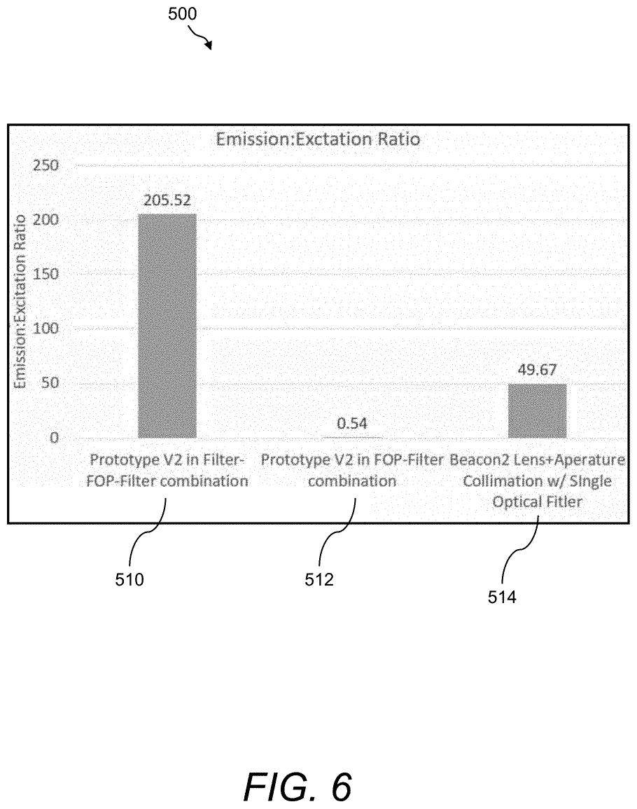

FIG. 6 illustrates an example of a bar graph 300 comparing the emission-to-excitation ratio of various optical filter configurations including the presently disclosed optical filter device 120. Bar 510 illustrates the performance of the optical filter device 220, shown and described with reference to FIG. 3. With a emission-to-excitation (signal-to-noise) ratio in excess of 200, FIG. 6 illustrates the dramatic performance improvement (more than 350 times better) than some known filter devices, that may only include two layers (bar 512). The experimental data for the an optical filter configuration including an FOP as a collimator to collimate the light prior to the light striking an optical bandpass filter, shown in bar 314 shows improved performance relative to non-collimated light, but inferior to the optical filter device 222 (bar 510).

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Some embodiment describe filtering as being “effective” or “ineffective.” In some instances, a filter is “effective” against (or “configured to reject”) a particular signal if it blocks >99.99% (10⁻⁴ rejection) of that signal. In other instances, an effective filter provides 10⁻⁵ or 10⁻⁶ rejection of out-of-band photons. Conversely, in some instances, a filter is ineffective against a particular signal if it allows more than 0.5%, 0.01%, 0.001% or 0.00001% of that signal to pass.

Although the foregoing subject matter has been described in some detail by way of illustration and example for purposes of clarity of understanding, it will be understood by those skilled in the art that certain changes and modifications can be practiced within the scope of the appended claims. Furthermore, although various embodiments have been described as having particular features and/or combinations of components, other embodiments are possible having a combination of any features and/or components from any of embodiments where appropriate as well as additional features and/or components.

Where methods described above indicate certain events occurring in certain order, the ordering of certain events may be modified. Additionally, certain of the events may be performed concurrently in a parallel process when possible, as well as performed sequentially as described above. Although various embodiments have been described as having particular features and/or combinations of components, other embodiments are possible having a combination of any features and/or components from any of the embodiments where appropriate.

That which is claimed:

1. An apparatus, comprising:
one or more optical bandpass filters; and
one or more angular filters,
the apparatus including at least three layers, each layer alternating between an optical bandpass filter from the one or more optical bandpass filters and an angular filter from the one or more angular filters.

2. The apparatus of claim 1, further comprising:
a light source configured to emit an excitation signal in an excitation wavelength range,
the one or more optical bandpass configured to reject the excitation wavelength range.

3. The apparatus of claim 1, further comprising:
a light source configured to emit an excitation signal in an excitation wavelength range,
the one or more optical bandpass filters configured to reject the excitation wavelength range at a zero degree angle of incidence.

4. The apparatus of claim 1, wherein:
the apparatus has at least three layers; and
an angular filter from the one or more angular filters is disposed between a first optical bandpass filter from the one or more optical bandpass filters and a second optical bandpass filter from the one or more optical bandpass filters.

5. The apparatus of claim 1, wherein:
the apparatus has at least three layers; and
an optical bandpass filter from the one or more optical bandpass filters is disposed between a first angular filter from the one or more angular filters and a second angular filter from the one or more angular filters.

6. The apparatus of claim 1, further comprising:
a light source configured to emit an excitation signal in an excitation wavelength range,
the one or more optical bandpass filters configured to reject the excitation wavelength range at a zero degree angle of incidence, the one or more optical bandpass filters ineffective to reject the excitation wavelength range at an angle of incidence greater than 30 degrees.

7. The apparatus of claim 1, further comprising:
a detector configured to measure a signal emitted in an emission wavelength range, the signal emitted in response to a sensor being illuminated by light in an excitation wavelength range,
the one or more optical bandpass are configured to reject the excitation wavelength range and pass the emission wavelength range.

8. The apparatus of claim 1, further comprising:
a detector configured to measure a signal emitted in an emission wavelength range, the signal emitted in response to a sensor being illuminated by light in an excitation wavelength range,
the one or more optical bandpass filters are configured to reject the excitation wavelength range at a zero degree angle of incidence.

9. The apparatus of claim 1, further comprising:
a detector configured to measure a signal emitted in an emission wavelength range, the signal emitted in response to a sensor being illuminated by light in an excitation wavelength range,
the one or more optical bandpass filters are configured to reject the excitation wavelength range at a zero degree angle of incidence, the one or more optical bandpass filters ineffective to reject the excitation wavelength range at an angle of incidence greater than 30 degrees.

10. The apparatus of claim 1, wherein:
The one or more optical bandpass filters are configured to reject the excitation wavelength range at a zero degree angle of incidence;
the one or more optical bandpass filters are configured to reject the excitation wavelength range at a zero degree angle of incidence, the one or more optical bandpass filters ineffective to reject the excitation wavelength range an at angle of incidence greater than 30 degrees; and
the one or more angular filters are configured to prevent excitation signal having an angle of incidence of greater than 20 degrees from reaching the second optical bandpass filter.

11. The apparatus of claim 1, wherein:
the apparatus has at least four layers; and
a first optical bandpass filter from the one or more optical bandpass filters is disposed between a first angular filter from the one or more angular filters and a second angular filter from the one or more angular filters; and
the second angular filter is disposed between the first optical bandpass filter and a second bandpass optical filter from the one or more optical bandpass filters.

12. The apparatus of claim 1, further comprising:
a light source configured to emit a first optical signal in an excitation band to illuminate a sensor embedded in a scattering matrix; and
a detector configured to detect a second optical signal in an emission band emitted from the sensor, an intensity of the second optical signal being at least an order of magnitude less than an intensity of the first optical signal,
the one or more optical bandpass filters being ineffective to reject back-scatter components of the first optical signal having an angle of incidence greater than 30 degrees,
the one or more angular filters configured to prevent back-scatter components of the first optical signal having an angle of incidence greater than 20 degrees from reaching the detector.

13. The apparatus of claim 1, further comprising:
a light source configured to emit a first optical signal in an excitation band to illuminate a sensor embedded in a scattering matrix; and
a detector configured to detect a second optical signal in an emission band emitted from the sensor, an intensity of the second optical signal being at least an order of magnitude less than an intensity of the first optical signal,
the one or more optical bandpass filters being ineffective to reject back-scatter components of the first optical signal having an angle of incidence greater than 30 degrees,
the one or more angular filters configured to prevent back-scatter components of the first optical signal having an angle of incidence greater than 20 degrees from reaching the detector,
the apparatus, configured such that an excitation-to-emission ratio at the detect or is at least 200.

14. The apparatus of claim 1, wherein the one or more optical bandpass filters and the one or more angular filters are an integrated optical filter constructed using silicon integrated circuit manufacturing techniques.

15. A method, comprising:
subjecting a diffuse optical signal to a first angular filter to produce a first filtered optical signal, the first angular filter configured to reject components of the diffuse optical signal that have an angle of incidence greater than 20 degrees;
subjecting the first filtered optical signal to a bandpass filter to produce a second filtered optical signal, the bandpass filter configured to reject components of the first filtered optical signal having an angle of incidence less than 30 degrees and a wavelength shorter than a predefined threshold;
subjecting the second filtered optical signal to a second angular filter to produce a third filtered optical signal, the second angular filter configured to reject components of the first filtered optical signal having an angle of incidence greater than 20 degrees.

16. The method of claim 15, wherein the bandpass filter is a first bandpass filter and the threshold is a first predefined threshold, the method further comprising:
subjecting the third filtered optical signal to a second bandpass filter to produce a fourth filtered optical signal, the second bandpass filter configured to reject components of the third filtered optical signal having a wavelength shorter than a second predefined threshold.

17. The method of claim 16, wherein the first predefined threshold and the second predefined threshold are the same predefined threshold.

18. The method of claim 15, further comprising illuminating a sensor with an excitation optical signal, the sensor disposed in a scattering environment such that the scattering environment produces at least a portion of the diffuse optical in an excitation band, a wavelength in the excitation band being shorter than the predefined threshold.

19. The method of claim 15, further comprising illuminating a sensor with an excitation optical signal, the sensor disposed in a scattering environment such that the scattering environment produces a first portion of the diffuse optical in an excitation band, a wavelength in the excitation band being shorter than the predefined threshold, the sensor configured to emit a second portion of the diffuse optical signal, the second portion of the diffuse optical signal being in an emission band, a wavelength in the emission band being longer than the predefined threshold.

20. The method of claim 15, further comprising:
illuminating a sensor with an excitation optical signal, the sensor disposed in a scattering environment such that the scattering environment produces a first portion of the diffuse optical in an excitation band, a wavelength in the excitation band being shorter than the predefined threshold, the sensor configured to emit a second portion of the diffuse optical signal, the second portion of the diffuse optical signal being in an emission band, a wavelength in the emission band being longer than the predefined threshold; and
detecting the second portion of the diffuse optical signal after subjecting the second filtered optical signal to the second angular filter.

21. The method of claim 15, further comprising illuminating a sensor with an excitation optical signal, the sensor disposed in a scattering environment such that the scattering environment produces at least a portion of the diffuse optical in an excitation band, a wavelength in the excitation band being shorter than the predefined threshold, a backscattered component of the excitation optical makes up less than 0.5% of the third filtered optical signal.

22. The method of claim 15, wherein the first angular filter is configured to reject components of the diffuse optical signal having an angle of incidence less than −20 degrees.

23. A method, comprising:
subjecting an optical signal to a first bandpass filter to produce a first filtered optical signal, the first bandpass filter configured to reject components of the diffuse optical signal that have an angle of incidence less than 30 degrees and a wavelength shorter than a first predefined threshold;
subjecting the first filtered optical signal to an angular filter to produce a second filtered optical signal, the angular filter configured to reject components of the first filtered optical signal having an angle of incidence greater than 20 degrees; and
subjecting the second filtered optical signal to a second bandpass filter to produce a third filtered optical signal, the second filtered optical signal configured to reject components of the second filtered optical signal having a wavelength shorter than a second predefined threshold.

24. The method of claim 23, wherein the optical signal is a diffuse optical signal.

25. The method of claim 23, wherein the angular filter is a first angular filter, the method further comprising:
subjecting a diffuse optical signal to a second angular filter to produce the optical signal, the second angular filter configured to reject components of the diffuse optical signal having an angle of incidence greater than 20 degrees.

26. The method of claim 23, further comprising illuminating a sensor with an excitation optical signal, the sensor disposed in a scattering environment such that the scattering environment produces at least a portion of the diffuse optical in an excitation band, a wavelength in the excitation band being shorter than the first predefined threshold and the second predefined threshold.

27. The method of claim 23, further comprising illuminating a sensor with an excitation optical signal, the sensor disposed in a scattering environment such that the scattering environment produces a first portion of the diffuse optical in an excitation band, a wavelength in the excitation band being shorter than the first predefined threshold and the second predefined threshold, the sensor configured to emit a second portion of the diffuse optical signal, the second portion of the diffuse optical signal being in an emission band, a wavelength in the emission band being longer than the first predefined threshold and the second predefined threshold.

28. The method of claim 23, further comprising:
illuminating a sensor with an excitation optical signal, the sensor disposed in a scattering environment such that the scattering environment produces a first portion of the diffuse optical in an excitation band, a wavelength in the excitation band being shorter than the first predefined threshold and the second predefined threshold, the sensor configured to emit a second portion of the diffuse optical signal, the second portion of the diffuse optical signal being in an emission band, a wavelength in the emission band being longer than the first predefined threshold and the second predefined threshold; and
detecting the second portion of the diffuse optical signal after subjecting the second filtered optical signal to the second bandpass filter.

29. The method of claim 23, wherein the first predefined threshold and the second predefined threshold are the same predefined threshold.

30. The method of claim 23, further comprising illuminating a sensor with an excitation optical signal, the sensor disposed in a scattering environment such that the scattering environment produces at least a portion of the diffuse optical in an excitation band, a wavelength in the excitation band being shorter than the first predefined threshold and the second predefined threshold, a backscattered component of the excitation optical makes up less than 0.5% of the third filtered optical signal.

31. The method of claim 23, wherein a filter window of the first bandpass filter is configured to be blueshifted for a component of the diffuse optical signal having an angle of incidence of greater than 30 degrees such that the first bandpass filter is configured to pass the component of the diffuse optical signal having a wavelength longer than the first predefined threshold such that the component forms a portion of the second filtered optical signal.

32. The method of claim 23, wherein the angular filter is configured to reject components of the first filtered optical signal having an angle of incidence less than −20 degrees.
